(19) United States
(12) Patent Application Publication
(10) Pub. No.: US 2004/0048272 A1
(43) Pub. Date: Mar. 11, 2004

(54) Dendritically amplified detection method

(76) Inventors: Itamar Willner, Mevasseret Zion (IL); Fernando Patolsky, Jerusalem (IL)

Correspondence Address: Nath & Associates, Sixth Floor, 1030 15th Street N W, Washington, DC, 20005, US

(21) Appl. No.: 10/381,131

(22) PCT Filed: Sep. 24, 2001
(86) PCT No.: PCT/IL01/00886

(30) Foreign Application Priority Data
Oct. 12, 2000 (IL) 138988

(51) Int. Cl. C12Q 1/68

(52) U.S. Cl. 435/6

(57) ABSTRACT

A method and system for the detection of a target nucleic acid in a sample solution. The target nucleic acid comprises a first and a second end sequence, one of the end sequences being a 5′ end sequence and the other end sequence being a 3′ end sequence. The method comprises: (a) attaching to a solid surface a first oligonucleotide probe, at least a portion of which is complementary to the first end sequence of the target nucleic acid; (b) contacting the solid surface with the sample solution, thereby allowing the first probe to bind the target nucleic acid; (c) providing a second semiconductor nanoparticle to which has been attached a second oligonucleotide probe, at least a portion of which is complementary to the second end sequence of the target nucleic acid; (d) contacting the solid surface of step (b) with the second nanoparticle, thereby allowing the second probe to bind the bound target nucleic acid; (e) providing a first semiconductor nanoparticle to which has been attached the first oligonucleotide probe and pre-incubating the first nanoparticle with the target nucleic acid, thereby allowing the first probe to bind the target nucleic acid; (f) contacting the solid surface of step (d) with the pre-incubated first nanoparticle, thereby allowing the target nucleic acid bound to the first probe to bind the second probe on the second nanoparticle; and (g) detecting the presence of the nanoparticles on the solid surface, thereby detecting the target nucleic acid.

### FIELD OF THE INVENTION

This invention relates to a method and system for detecting nucleic acids.

### BACKGROUND OF THE INVENTION

The following references are referred to in the specification by number:

1. Kelley, S. O. & Barton, J. K. Electron transfer between bases in double helical DNA Science 283, 375-381 (1999).

2. Ratner, M. Photochemistry: Electronic motion in DNA. Nature 397, 480-481 (1999).

3. Braun, E., Eichen Y., Sivan, U. & Ben-Yoseph, G. DNA-templated assembly and electrode attachment of a conducting silver wire. Nature 391, 775-778 (1998).

4. Porath, D., Bezryadin, A., de Vries, S., Dekker, C. Direct measurement of electrical transport through DNA molecules. Nature 403, 635-638, (2000).

5. Storhoff, J. J. & Mirkin, C. A. Programmed materials synthesis with DNA. Chem. Rev. 99, 1849-1862 (1999).

6. Seeman, N. C. Nucleic acid nanostructures and topology. Angew. Chem. Int. Ed. Engl. 37, 3220-3238 (1998).

7. Takenaka, S., Yamasshita, K., Takagi, M., Uto, Y. & Kondo, H. DNA sensing on a DNA probe-modified electrode using ferrocenyl naphthalene diimide as the electrochemicaly active ligand Anal. Chem. 72(6), 1334-1341 (2000).

8. Korri-Yousoufi, H., Garnier, F., Srivtava, P., Godillot, P. & Yassow, A. Toward bioelectronics; specific DNA recognition based on digonucleotide-functionalized polypyrrole. J. Am. Chem. Soc. 119, 7388-7389 (1997).

9. Patolsky, F., Katz, E., Bardea, A. & Willner, I. Enzyme-linked electrochemical sensing of oligonucleotide-DNA in interactions by means of impedance spectroscopy. Langmuir 15, 3703-3706 (1999).

10. Patolsky, F., Ranjit, K. T., Lichtenstein, A. & Willner, I. Dendritic amplification of DNA analysis by oligonucleotide functionalized Au-nanoparticles. Chem. Commun. 1025-1026(2000).

11. Torimoto, T., Yamashita, M., Kuwabata, S., Sakata, T., Mori, H. and Yoneyama, H. Fabrication of CdS nanoparticle chain along DNA double strands. J. Phys. Chem. B 103(42): 8799-8803 (1999).

DNA-based electronics has been the subject of extensive recent research activities that address the conductivity features of double-stranded (ds) DNA [1,2]. The use of ds-DNA as a template for the construction of nanowires [3], and the use of metal-nanoparticles crosslinked by DNA as single-electron charging devices [4] has been described. The optical properties of DNA-crosslinked Au-nanoparticles were recently studied and applied for DNA sensing [5], and nano-architectures of DNA/Au-nanoparticles were assembled [6]. The electronic transduction of DNA sensing, and specifically the amplified DNA analyses, were recently reported by the use of electrochemical [7] or microgravimetric quartz-crystal-microbalance measurements. Direct electrochemical detection of DNA was reported by following the electrochemical response of DNA [8] or the incorporation of redox-labels into ds-DNA The amplified detection of DNA was accomplished by the use of biocatalytic conjugates [9] or the application of labeled liposomes or nanoparticles [10].

CdS nanoparticle chains have been fabricated along ds DNA by depositing DNA on a lipid monolayer and subsequently adding CdS nanoparticles. The nanoparticles formed a chain on the DNA template due to the electrostatic interaction between cationic surface modifiers on the nanoparticle surface and the phosphate groups of the DNA [11].

### SUMMARY OF THE INVENTION

It is an object of the present invention to provide a method and system for detecting target nucleic acids in a sample.

The terms “detect” or “detection” in this specification refer collectively to both a qualitative determination and identification of the target nucleic acid in the sample as well as, at times, a quantitative determination of the level of the target nucleic acid in the sample.

The present invention provides a method for constructing a dendritic architecture of double-stranded nucleic acid crosslinked seniconductor-nanoparticle arrays on solid supports and the structurally-controlled generation of photocurrents and/or optical signals upon irradiation of these arrays.

In one embodiment of a first aspect, the present invention provides a method for the detection of a target nucleic acid in a sample solution, said target nucleic acid comprising a first and a second end sequence, one of said end is sequences being a 5′ end sequence and the other end sequence being a 3′ end sequence, said method comprising:

(a) providing a solid surface;

(b) attaching to said solid surface a first oligonucleotide probe, at least a portion of which is complementary to the first end sequence of said target nucleic acid;

(c) contacting the solid surface of step (b) with said sample solution, thereby allowing said first probe to bind said target nucleic acid;

(d) providing a second semiconductor nanoparticle to which has been attached a second oligonucleotide probe, at least a portion of which is complementary to the second end sequence of said target nucleic acid;

(e) contacting the solid surface of step (c) with said second nanoparticle, thereby allowing said second probe to bind said bound target nucleic acid;

(f) providing a first semiconductor nanoparticle to which has been attached said first oligonucleotide probe and pre-incubating said first nanoparticle with said target nucleic acid, thereby allowing said first probe to bind said target nucleic acid,

(g) contacting the solid surface of step (e) with said pre-incubated first nanoparticle, thereby allowing said target nucleic acid bound to said first probe to bind said second probe on said second nanoparticle;

(h) optionally alternately repeating steps (e) and (g) one or more times; and

(i) detecting the presence of said nanoparticles on said solid surface, thereby detecting said target nucleic acid.

In the above embodiment of the invention, the target nucleic acid (in the sample solution) is first contacted with the immobilized oligonucleotide probe on the solid surface. In an alternate embodiment of this aspect of the invention, the target nucleic acid is first contacted with the immobilized oligonucleotide probe on the nanoparticle. Thus, this alternate embodiment is performed as follows:

(a) providing a solid surface;

(b) attaching to said solid surface a first oligonucleotide probe, at least a portion of which is complementary to the first end sequence of said target nucleic acid;

(c) providing a second semiconductor nanoparticle to which has been attached a second oligonucleotide probe, at least a portion of which is complementary to the second end sequence of said target-nucleic acid and pre-incubating said second nanoparticle with said target nucleic acid, thereby allowing said second probe to bind said target nucleic acid;

(d) contacting the solid surface of step (b) with said pre-incubated second nanoparticle, thereby allowing said bound target nucleic acid to bind said first probe;

(e) providing a first semiconductor nanoparticle to which has been attached said first oligonucleotide probe;

(f) contacting the solid surface of step (d) with said first nanoparticle, thereby allowing said target nucleic acid bound to said second probe to bind said first probe on said first nanoparticle;

(g) optionally alternately repeating steps (d) and (f) one or more times; and

(h) detecting the presence of said nanoparticles on said solid surface, thereby detecting said target nucleic acid.

The nanoparticle used in the method of the invention may comprise any semiconducting compound having photoconductive properties. Examples of such compounds include CdS, CdSe, GaAs, PbS and ZnS. CdS is a preferred nanoparticle compound. The nanoparticles in one array may comprise the same or different semiconducting compounds. In a preferred embodiment, the nanoparticles comprise the same semiconducting compound.

The presence of the nanoparticles may be detected optically or photoelectrochemically.

If the nanoparticles are detected optically, this may be by any technique known per se, such as fluorescence detection or light absorbance detection In this case, the solid surface on which the array is fabricated may be any material to is which an oligonucleotide may be bound either directly or indirectly. Examples of such materials include a glass or polymer support

If, on the other hand, the nanoparticles are detected photoelectrochemically, the solid support must be an electrode which can sense the photocurrent produced by irradiation of the nanoparticles. A non-limiting example of such an electrode is an Au-electrode. The nanoparticles may be detected by measuring current. flows or voltage. The detected signal may be amplified by incubating the electrode with an electron mediator capable of binding nucleic acids The electrostatic binding of the electron mediator on the nucleic acid units may provide tunneling routes for the conduction-band electrons, resulting in an enhanced photocurrent Examples of such electron mediators include organic compounds, transition metal complexes or metallic nanorods which can associate by electrostatic binding and/or intercalate with nucleic acids, thus improving the electrical contacting of the semiconductor nanoparticles with the electrode.

The term “nucleic acid” in the present specification includes both DNA and RNA. The oligonucleotide probe will typically, but not exclusively, comprise a number of nucleotides completing about one helix of the nucleic acid stand, i.e. about twelve nucleotides. A sequence of twelve oligonucleotides ensures, on the one hand, stable hybridization and, on the other hand, a 12-mer oligonucleotide decreases the chance of binding to an incorrect nucleic acid than in the case of a longer sequence. In the case where the sample is a digested specimen of genomic DNA, or a fractionation product thereof comprising the nucleic acids, there is some probability, which increases with the length of the capturing oligonucleotide, of binding to an incorrect oligonucleotide, namely an oligonucleotide other than the target oligonucleotide. This probability is lower, as aforesaid in the case of a shorter oligonucleotide. On the other hand, the specificity of binding increases with the length of the oligonucleotide with respect to longer target molecules. A sequence of about 12 nucleotides is preferred as it is optimal as far as ensuring binding stability, on the one hand, and reducing incorrect binding on the other hand. The invention is, however, not limited to such a length of the oligonucleotide probe, and the skilled man of the art will know how to adjust the length of the probe to the requirements of the method.

In a second aspect of the invention, there is provided a method for fabricating a multi-layered array of semiconductor nanoparticles crosslinked by nucleic acid comprising the steps of the method of the first aspect of the invention in both of its embodiments.

In a third aspect of the invention, there is provided a method for fabricating a semiconductor nanoparticle electronic circuit comprising electron mediator functionalized nucleic acid comprising:

(a) providing an electrode;

(b) attaching to said electrode a first oligonucleotide probe, at least a portion of which is complementary to a first end sequence of a nucleic acid;

(c) contacting the electrode of step (b) with said nucleic, acid, thereby allowing said first probe to bind said nucleic acid;

(d) providing a second semiconductor nanoparticle to which has been attached a second oligonucleotide probe, at least a portion of which is complementary to a second end sequence of said nucleic acid;

(e) contacting the electrode of step (c) with said second nanoparticle, thereby allowing said second probe to bind said bound nucleic acid;

(f) providing a first semiconductor nanoparticle to which has been attached said first oligonucleotide probe and pre-incubating said first nanoparticle with said nucleic acid, thereby allowing said first probe to bind said nucleic acid;

(g) contacting the electrode of step (e) with said pre-incubated first nanoparticle, thereby allowing said nucleic acid bound to said first probe to bind said second probe on said second nanoparticle;

(h) optionally alternately repeating steps (e) and (g) one or more times; and

(i) incubating said electrode with an electron mediator capable of binding nucleic acids.

An alternate embodiment of the third aspect of the invention provides a method for fabricating a semiconductor nanoparticle electronic circuit comprising semiconductor arrays crosslinked by nano metallic rods in which the last step comprises incubating said electrode with a metal capable of binding nucleic acids.

Also contemplated by the invention a semiconductor device comprising. a dendritic nanoparticle array comprising semiconductor nanoparticles cross-linked by nucleic acid chains.

In a fourth aspect of the invention, there is provided a system for identifying a target nucleic acid sequence in a sample solution comprising:

(a) a biochip comprising a plurality of arrays of functionalized solid surfaces each of which may act as a transducer, each of the surfaces having bound thereto an oligonucleotide probe, at least a portion of which is complementary to a different segment of a target nucleic acid sequence, each of the arrays being specific for a different target nucleic acid sequence; and.

(b) semiconductor nanoparticles functionalized with oligonucleotide probes, at least a portion of which is complementary to one end sequence or the other end sequence of one of the target nucleic acid sequences.

In this aspect of the invention, parallel analysis of multiple samples may be carried out on microarrays of functionalized solid surfaces. For example, if it is desired to determine the identity of a infecting pathogenic microorganism such as a virus in a sample, a DNA chip or bio-chip may be used in which one row of solid surfaces will comprise probes complementary to different segments of the genetic material of one type of virus, a second row will comprise probes complementary to a second type of virus, etc. Application of the sample to the biochip, contacting it with the functionalized semiconductor nanoparticles and locating the row which produces a signal will enable identification of the infecting virus. A similar detection system may be used to identify genetic mutants and diseases, in tissue typing, gene analysis and forensic applications.

In a fifth aspect of the invention, there is provided a kit for the detection of a target nucleic acid sequence in a sample containing a mixture of nucleic acids comprising:

(a) a functionalized solid surface which acts as a transducer and having a probe attached thereto; and

(b) semiconductor nanoparticles functionalized with oligonucleotide probes, a portion of which is complementary to one end or the other end of the target nucleic acid sequence.

### BRIEF DESCRIPTION OF THE DRAWINGS

In order to understand the invention and to see how it may be carried out in practice, preferred embodiments will now be described, by way of non-limiting example only, with reference to the accompanying drawings, in which:

FIG. 1 is a schematic drawing illustrating the organization of oligonucleotide/DNA crosslinked arrays of CdS-nanoparticles according to one embodiment of the invention and the photoelectrochemical response of the nanoarchitectures;

FIG. 2 is a schematic drawing illustrating an alternate embodiment of the method of the invention;

FIG. 3 shows the frequency change of an Au/quartz crystal (9 MHz, AT-cut upon the assembly of oligonucleotide/DNA crosslinked CdS-nanoparticle layers: the first layer is assembled by the reaction of the (1)-functionalized electrode with (3), 1×10⁻⁶ M, and then with the (2)-modified CdS nanoparticles. The other layers were constructed by the alternate treatment of the surface with a solution of (3), 1×10⁻⁶ M that includes the (1)-modified CdS nanoparticles and a solution of (2)-functionalized CdS-nanoparticles;

FIG. 4 shows the absorbance spectra (I) and fluorescence spectra (II) of layered oligonucleotide/DNA crosslinked CdS nanoparticle arrays: (a) to (d) correspond to (1) to (4) CdS nanoparticle layers, λₑₓ=405 nm for fluorescence spectra;

FIG. 5 shows photocurrent action spectra of an Au-electrode that includes programmed layers of oligonucleotide/DNA crosslinked CdS nanoparticles: (a) Prior to the deposition of CdS-nanoparticles. (b) to (e) One to four oligonucleotide/DNA crosslinked CdS nanoparticle layers. Inset: Comparison of the photocurrent action spectrum of a four-layer CdS nanoparticle array (e) to the absorption spectrum (f) of the array;

FIG. 6 shows photocurrent action spectra of: two-layer (a) and four layer (c) oligonucleotide CdS-nanoparticle crosslinked arrays. A two-layer (b) and a four-layer 9d) oligonucleotide/DNA CdS-nanoparticle crosslinked arrays in the presence of Ru(NH₃)₆³⁺, 5×10⁻⁶ M. All photocurrent spectra were recorded under argon in 0.1 M KCl using triethanolamine, 2×10⁻² M as sacrificial electron donor. The area of illuminated electrode corresponds to 1 cm²; and

FIG. 7shows sensing of the DNA (3) by the photocurrent response of the arrays. The photocurrent responses of: (a) A two-layer oligonucleotide/DNA CdS crosslinked array. (b) Upon treatment of the two-layer crosslinked array with (3), 1×10⁻⁹ M, in the presence of (2)-functionalized CdS. (c) Upon the treatment of the two-layer crosslinked array with (3), 1×10⁻⁸ M, in the presence of (2)-functionalized CdS. (d) Upon the treatment of the two-layer crosslinked array with (3), 1×10⁻⁷ M in the presence of (2)-functionalized CdS. Photocurrent spectra were recorded at the conditions specified in the caption of FIG. 5.

### DETAILED DESCRIPTION OF THE INVENTION

Methods and Materials

I. Preparation of Q-CdS nanoparticles

A 0.24 ml aliquot of a 1.0 M Cd(CIO₄)₂ aqueous solution and 0.16 ml of a 1.0 Na₂S aqueous solution were respectively added to 60 and 40 ml aliquots of the prepared inverse micelle solution. After the solution was stirred individually for 1 hr, these were mixed together and stirred for another 1 hr, resulting in the formation of Q-CdS in the inverse micelles.

The surface of the resulting Q-CdS was modified both with 2-aminoethanethiol and with 2-mercapto ethanesulfonate. The modification with the latter compound was essential to dissolve the resulting particles in water solutions later. Both 0.17 mL of an 0.32 M 2-aminoethanethiol aqueous solution and 0.33 mL 0.32 M 2-mercapto ethanesulfonate solution were added to 100 mL of the inverse micelles solution containing !-CdS and stirred for 1 day, under Ar atmosphere, resulting in thiol-capped Q-CdS nanoparticles. After drying under vacuum, the thiol-capped QCdS was washed successively with pyridine, n-heptane, petroleum ether, 1-butanol, acetone, and methanol.

II. Preparation of DNA-modified Q-CdS nanoparticles:

Procedure 1: Thiol exchange procedure:

To a Q-CdS aqueous solution (1 mg/mL), a reduced thiolated-DNA solution (8-10 OD/mL) was added, and stirred. After standing for 24 hr at room temperature, the solution was brought to 0.2 M NaCl, 0.1 M phosphate buffer pH=7.4. after standing for an additional 24 hrs, the solution was dialyzed against 0.2 M NaCl+0.1 M phosphate buffer, pH=7.4+0.01% sodium azide over a period of 2-3 days at 4° C., during which time the dialyzing solution was refreshed 4-7 times. The DNA modified Q-CdS nanoparticles could be kept in solution for further experiments, and could also be obtained as a solid compound by using speed-back.

Procedure 2: Chemical-binding of DNA to the Q-CdS surfaces

To a thiol-modified Q-CdS solution (1 mg/mL), pH=7.4, an excess amount of the crosslinker 4-(N-maleimidomethyl)cyclohexane-1-carboxilic acid 3-sulfo-N- hydroxy-succinimide ester was added, and the reaction carried out for a period of 24 hrs at 4° C., followed by centrifugation for at least 1 hr at 20,000 rpm to remove excess reagents. Following removal of the supernatant, the yellow precipitate was washed with 0.1 M NaCl, 0.1 M phosphate buffer, pH=7.4, and recentrifuged. This procedure was repeated at least 4 times.

To a solution of chemically-reactive maleimide functionalized Q-CdS nanoparticles, a buffer solution containing the thiolated DNA (10 )D/mL) was added, and aged for a period of 12 hrs, at 4° C. After that the excess thiolated oligonucleotides were removed by centrifugation at 20,000 rpm, 1 hr at least, or by dialysis against PBS buffer, containing 0.02% NaN₃, for 2 days at 4° C. The DNA-modified Q-CdS nanoparticles obtained by the two procedures outlined here were extremely soluble in water solutions, compared with the regular Q-CdS nanoparticles before DNA modification.

### EXAMPLES

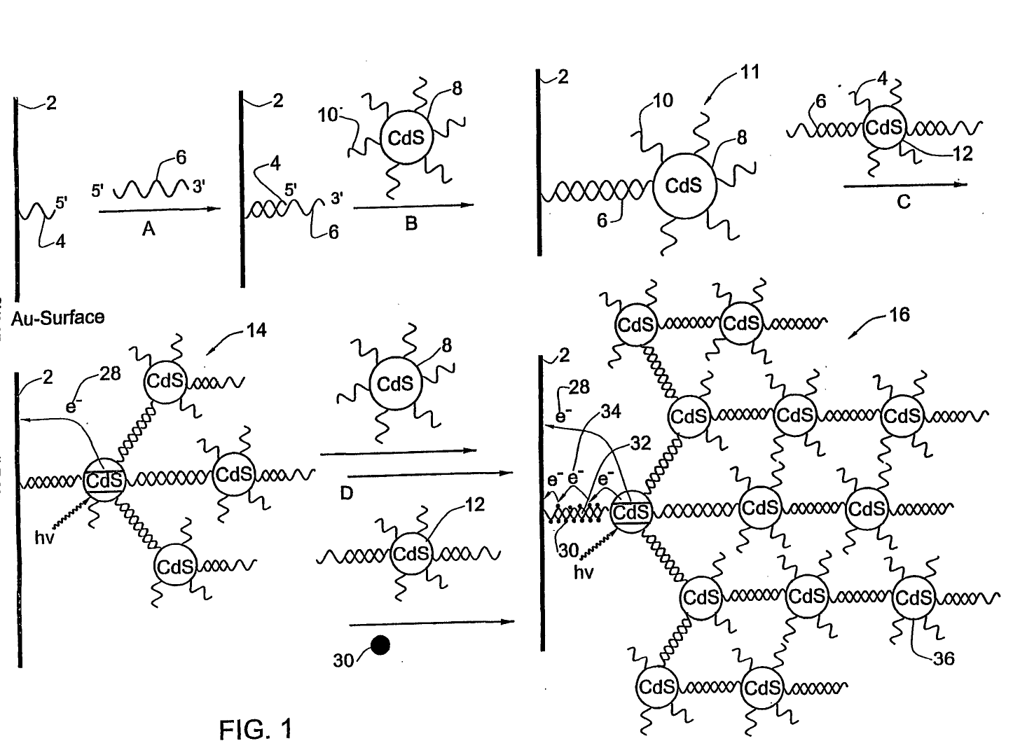

One embodiment of the method of the invention is depicted schematically in FIG. 1. The following oligonucleotides were used as probes or target in the following examples:

| | |
|---|---|
| (4) | |
| 5′TCTATCCTACGCT-(CH₂)₆-SH-3′ | (SEQ ID NO:1) |
| (10) | |
| 5′-HS-(CH₂)₆-GCGCGAACCGTATA-3′ | (SEQ ID NO:2) |
| (6) | |
| 5′-AGCGTAGGATAGATATACGGTTCGCGC-3′ | (SEQ ID NO:3) |
| 5′-AGCGCTCCAGTGATATACGGTTCGCGC-3′ | (SEQ ID NO:4) |

### Example I

FIG. 1 illustrates the stepwise assembly of the DNA-crosslinked CdS particles on a solid surface in the form of a Au-electrode 2. A first oligonucleotide probe 4 (e.g. SEQ ID NO:1) is complementary to the 5′ end of a target DNA 6 (SEQ ID NO: 3). The first probe 4 is attached to an Au-electrode 2 (2.3×10⁻¹¹ mole·cm⁻²), and the electrode is then interacted in reaction A with the sample solution containing the target DNA 6 to yield the ds-system.

CdS-nanoparticles (2.6±0.4 nm) were functionalized with the thiolated first and second oligonucleotide probes 4 or 10. These two oligonucleotides are complementary to the 5′ and 3′-ends of the target DNA 6, respectively. In reaction B, the electrode 2 is contacted with the second oligonucleotide probe 10 (SEQ ID NO: 2) functionalized nanoparticles 8 resulting in the binding of the CdS-nanoparticles 8 to the target DNA 6 bound to the electrode 2. This is termed the first generation 11 of the nanoparticle array.

A further CdS nanoparticle 12 functionalized with, the first oligonucleotide probe 4 was pre-incubated (1 mg.ml⁻1) with the target DNA 6 (1×10⁻⁶ M), so that the target DNA bound to some of the probes 4 extending from the nanoparticle 12. The electrode 2 carrying the first nanoparticle generation was contacted in reaction C with the first probe-functionalized and target DNA- pre-incubated nanoparticles 12 resulting in the binding of the pre-incubated nanoparticles 12 to the first generation nanoparticles 8 This is termed the second generation 14 of the nanoparticle array.

Further alternate contacting of the electrode 2 with solutions consisting of the second probe 10 functionalized CdS nanoparticles 8 and the first probe 4 functionalized CdS-nanoparticles 12, results in an array with a controlled number of CdS-nanoparticle generations 16 (reaction D). It may be seen that the number of nanoparticles increases exponentially as a function of the number of generations, and forms a dendritic architecture. It will be clear that the fabrication of the array is only made possible by the presence of the target DNA. In this way, detection of the presence of the nanoparticle array is indicative of the presence of the target DNA.

### Example II

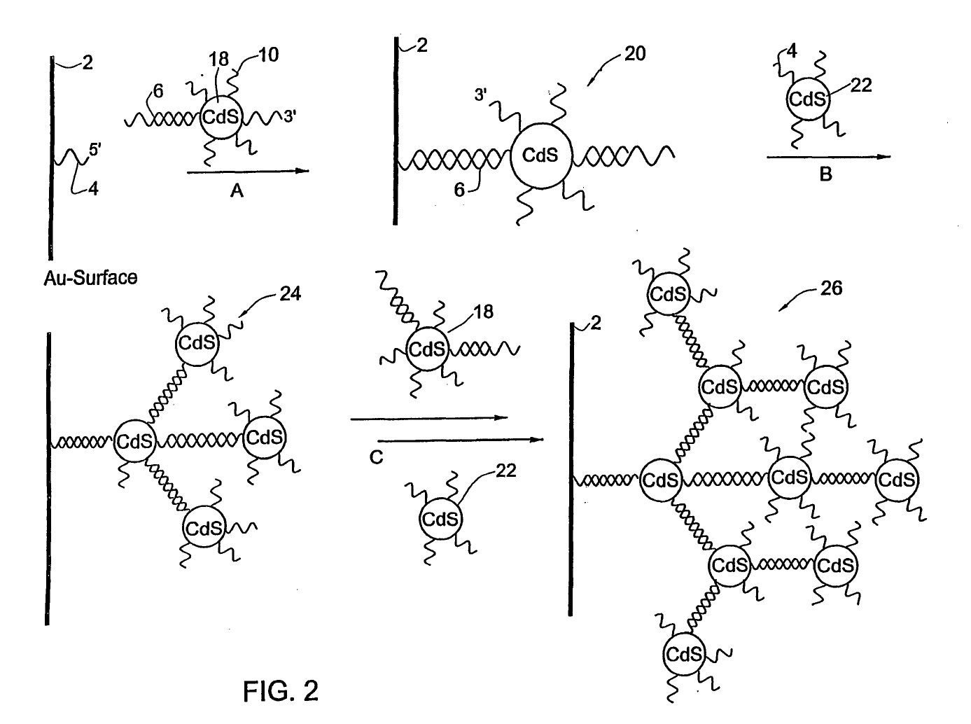

An alternate embodiment of the method of the invention is illustrated in FIG. 2. As before, the first probe 4 is attached to the Au-electrode 2. However, in this case, the second oligonucleotide probe 10-functionalized nanoparticles 18 are pre-incubated with the target DNA 6 so that the target DNA binds to some of the probes 10 extending from the nanoparticle 8. These pre-incubated nanoparticles 18 are contacted in reaction A with the electrode 2 so that the target DNA 6 bound to the nanoparticle binds to the immobilized first probe 4 on the electrode, resulting in the first generation 20. The electrode is then contacted in reaction B with a nanoparticle 22 functionalized with the first probe 4 which binds to the target DNA 6 forming the second generation 24. As before, these contacting steps are repeated alternately to generate the desired number of generations 26.

### Example III

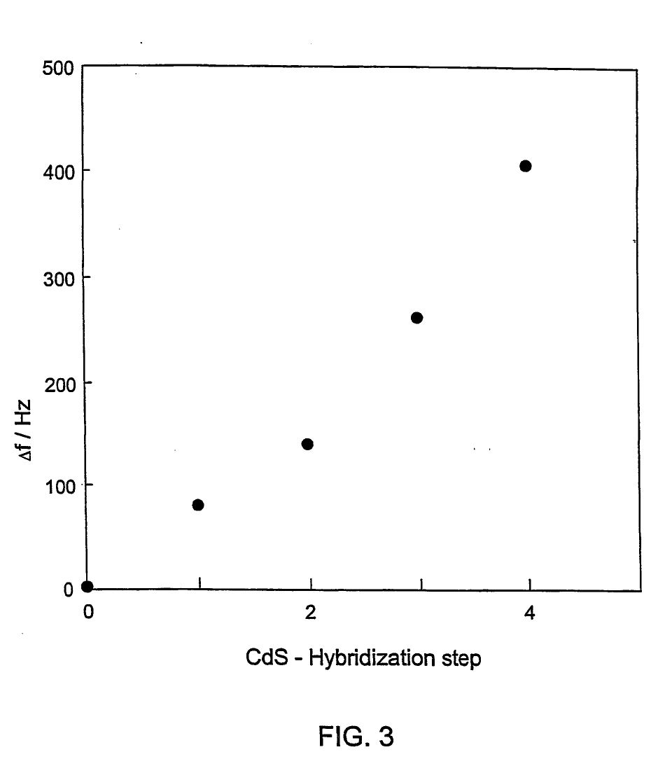

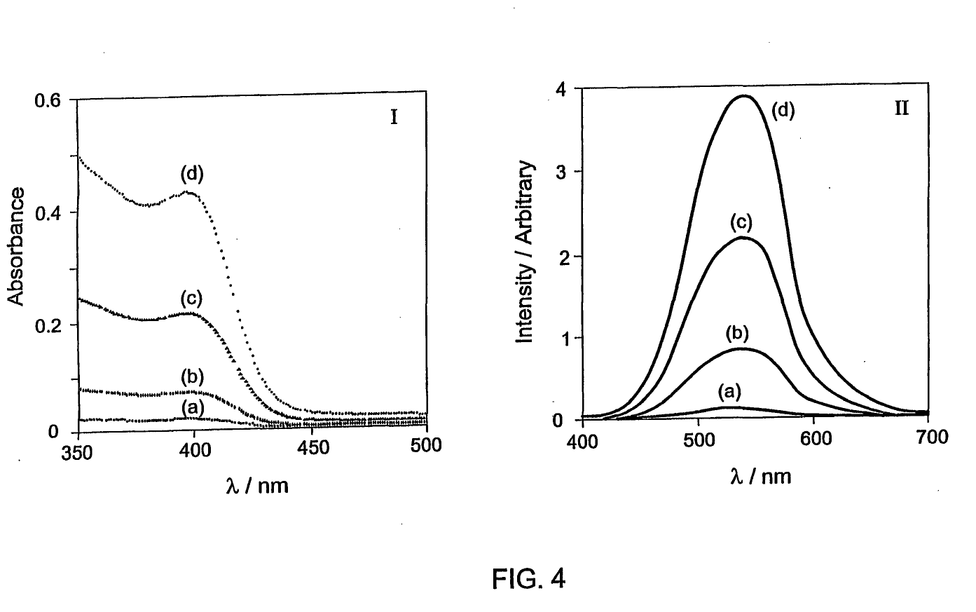

The build-up of the DNA-crosslinked CdS-nanoparticle array was followed by microgravimetric quartz-crystal- microbalance experiments, the results of which are shown in FIG. 3. Similarly, the DNA-crosslinked CdS-nanoparticle arrays were assembled on glass supports using an aminopropylsiloxane-functionalized glass that was reacted with ε-maleimidocaproic acid N-hydroxysuccinimide ester [10] as a base interface for the covalent age of the oligonucleotide probe and the organization of the nanoparticle systems. FIG. 4 shows the absorbance spectra and the fluorescence spectra corresponding to the DNA-crosslinked CdS-nanoparticle arrays. The absorbance and fluorescence spectra increase as the generation of aggregated CdS increases.

### Example IV

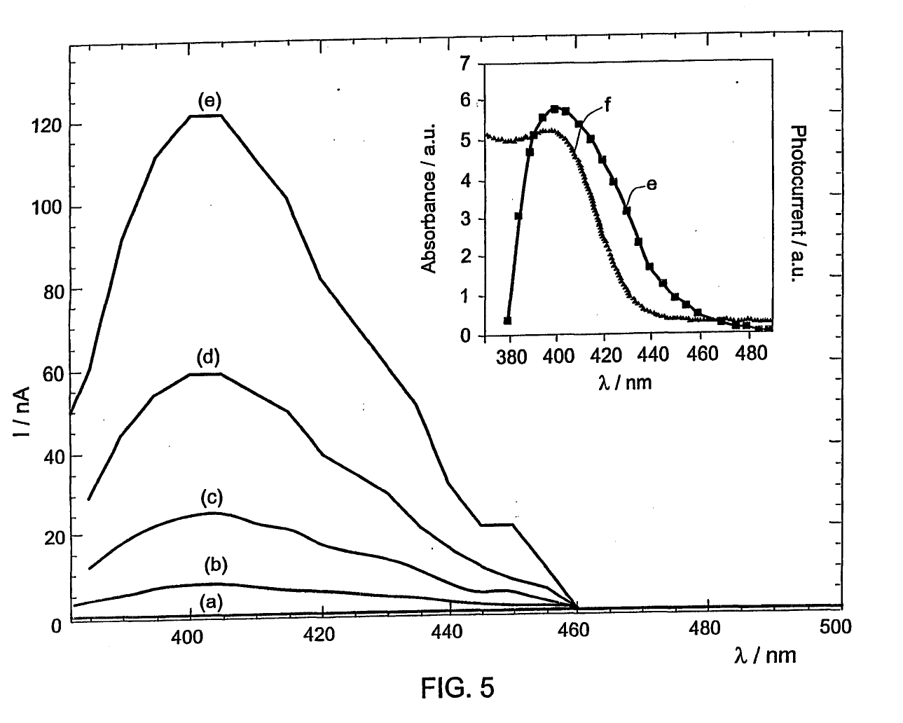

FIG. 5 shows the photocurrent action spectra upon the excitation of the arrays that consist of different numbers of CdS nanoparticle generations that are associated with the electrode. The photocurrent follows the absorbance spectrum of the CdS-nanoparticles (inset, FIG. 5), and it increases as the number of generations of crosslinked particles is higher. The photocurrent can be switched “ON” and “OFF” by pulsed irradiation of the respective arrays. The mechanism of photocurrent generation probably involves the photoejection of conduction-band electrons 28 of CdS-particles in contact or at tunneling distances from the electrode 2, as shown in FIG. 1. This suggests, however, that a part of the crosslinked crosslinked-nanoparticles do not participate in the development of the photocurrent.

To assist the generation of the photocurrent by CdS inactive particles and referring again to FIG. 1, the arrays 14 were reacted with an electron mediator 30 such as Ru(NH₃)₆³⁺, 5×10⁻⁶ M, that electrostatically binds to the DNA 32. The transition-metal complex, E°=−0.16 V vs. SCE, acts as an electron acceptor for the conduction-band electrons 34 (E°_{CB}=<−0.9V vs. SCE), and thus could mediate the electron transfer from remote, inactive CdS particles 36 to the electrode.

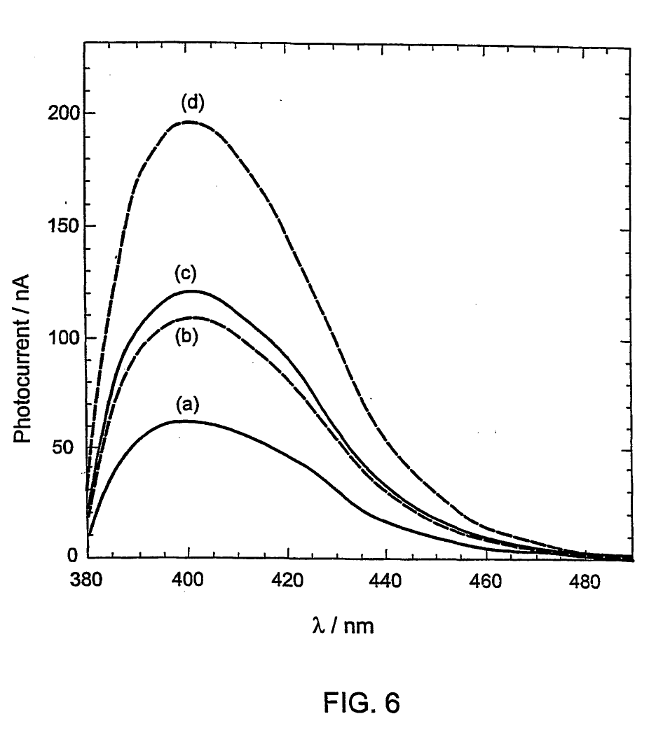

FIG. 6 shows the photocurrents that are generated by the DNA-crosslinked CdS arrays that include two and four CdS-nanoparticle generations in the absence and presence of Ru(NH₃)₆³⁺, respectively. In the presence of Ru(NH₃)₆³⁺ the photocurrent is ca. two-fold higher, implying that the DNA units act as a template for the electron acceptor units that mediate electron transfer to the electrode. It should be noted that the increase of the Ru(NH₃)₆³⁺ concentration to 5×10⁻⁴ M, adversely affects the photocurrent and it decreases to values below those observed in the presence of the CdS-arrays without the electron acceptor. This result is reasonable since at high bulk concentrations of Ru(NH₃)₆³⁺ diffusional electron transfer quenching of the semiconductor nanoparticles proceeds. This process traps the conduction-band electrons and thus prevents even the direct electron photoejection process.

### Example V

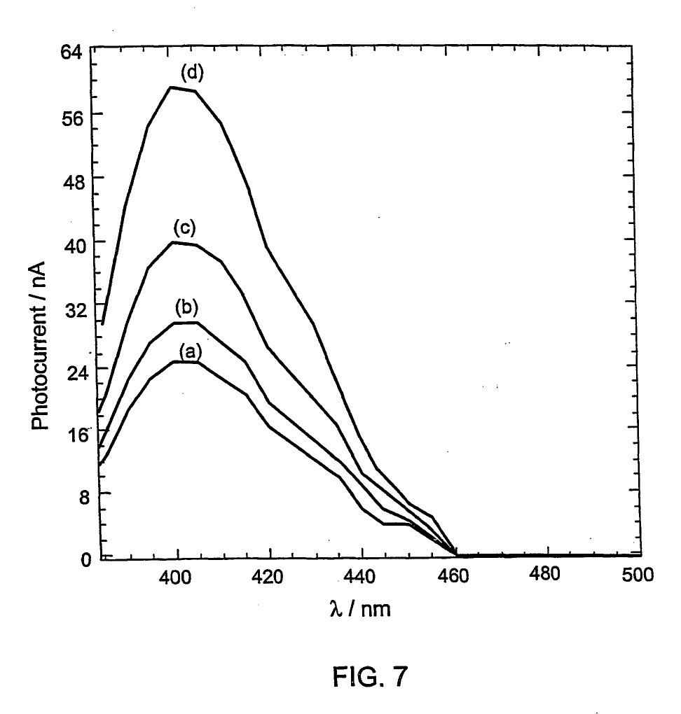

The photocurrents generated by the DNA-crosslinked array can be used for the quantitative detection of DNA. FIG. 7 shows the photocurrents of a two-layer DNA-crosslinked nanoparticle array upon the formation of a third generation of CdS-nanoparticles in the presence of probe-functionalized CdS at different concentrations of target nucleic acid. As the concentration of target nucleic acid is increased, enhanced photocurrents are observed, indicating higher coverage of the electrode by the third generation of semiconductor nanoparticles.

In all of the systems tested, no photocurrents were observed upon interaction of the probe-functionalized electrode with the probe-functionalized nanoparticles in the absence of target nucleic acid, or upon an attempt to crosslink the nanoparticle arrays with a non-specific oligonucleotide probe (e.g. SEQ ID NO: 4). Thus, no non-specific binding of the CdS-nanoparticles to the transducers is observed and the photocurrents are specific to the crosslinking process by the target nucleic acid.

1. A method for the detection of a target nucleic acid in a sample solution, said target nucleic acid comprising a first and a second end sequence, one of said end sequences being a 5′ end sequence and the other end sequence being a 3′ end sequence, said method comprising:
(a) providing a solid surface;
(b) attaching to said solid surface a first oligonucleotide probe, at least a portion of which is complementary to the first end sequence of said target nucleic acid;
(c) contacting the solid surface of step (b) with said sample solution, thereby allowing said first probe to bind said target nucleic acid;
(d) providing a second semiconductor nanoparticle to which has been attached a second oligonucleotide probe, at least a portion of which is complementary to the second end sequence of said target nucleic acid;
(e) contacting the solid surface of step (c) with said second nanoparticle, thereby allowing said second probe to bind said bound target nucleic acid;
(f) providing a first semiconductor nanoparticle to which has been attached said first oligonucleotide probe and pre-incubating said first nanoparticle with said target nucleic acid, thereby allowing said first probe to bind said target nucleic acid;
(g) contacting the solid surface of step (e) with said pre-incubated first nanoparticle, thereby allowing said target nucleic acid bound to said first probe to bind said second probe on said second nanoparticle;
(h) optionally alternately repeating steps (e) and (g) one or more times; and
(i) detecting the presence of said nanoparticles on said solid surface, thereby detecting said target nucleic acid.

2. A method according to claim 1 wherein said nanoparticle comprises a semiconducting compound selected from the group consisting of CdS, CdSe, GaAs, PbS and ZnS.

3. A method according to claim 1 wherein said nanoparticles comprise the same semiconducting compound.

4. A method according to claim 1 wherein said nanoparticles comprise different semiconducting compounds.

5. A method according to claim 1 wherein said nanoparticles are detected optically.

6. A method according to Claim 5 wherein said nanoparticles are detected by fluorescence detection or by light absorbance.

7. A method according to claim 1 wherein said solid surface comprises a glass or polymer support.

8. A method according to claim 1 wherein said nanoparticles are detected photoelectrochemically.

9. A method according to claim 8 wherein said nanoparticles are detected by measuring current flow or voltage.

10. A method according to either of claims 8 or 9 wherein said solid support is an electrode.

11. A method according to claim 8 further comprising before step (i) the step of:
(h1) incubating said solid surface with an electron mediator capable of binding nucleic acids.

12. A method according to claim 11 wherein said electron mediator is an organic compound, a transition metal complex or a metallic nanorod.

13. A method for the detection of a target nucleic acid in a sample solution, said target nucleic acid comprising a first and a second end sequence, one of said end sequences being a 5′ end sequence and the other end sequence being a 3′ end sequence, said method comprising:
(a) providing a solid surface;
(b) attaching to said solid surface a first oligonucleotide probe, at least a portion of which is complementary to the first end sequence of said target nucleic acid;
(c) providing a second semiconductor nanoparticle to which has been attached a second oligonucleotide probe, at least a portion of which is complementary to the second end sequence of said target nucleic acid and pre-incubating said second nanoparticle with said target nucleic acid, thereby allowing said second probe to bind said target nucleic acid;
(d) contacting the solid surface of step (b) with said pre-incubated second nanoparticle, thereby allowing said bound target nucleic acid to bind said first probe;
(e) providing a first semiconductor nanoparticle to which has been attached said first oligonucleotide probe;
(f) contacting the solid surface of step (d) with said first nanoparticle, thereby allowing said target nucleic acid bound to said second probe to bind said first probe on said first nanoparticle;
(g) optionally alternately repeating steps (d) and (f) one or more times; and
(h) detecting the presence of said nanoparticles on said solid surface, thereby detecting said target nucleic acid.

14. A method according to claim 13 wherein said solid support is an electrode.

15. A method according to claim 14 further comprising before step (h) the step of:
(g1) incubating said electrode with an electron mediator capable of binding nucleic acids.

16. A method according to claim 15 wherein said electron mediator is an organic compound, a transition metal complex or a metallic nanorod.

17. A method according to claim 1 wherein said nucleic acid is DNA or RNA.

18. A method for fabricating a multi-layered array of semiconductor nanoparticles crosslinked by nucleic acid comprising:
(a) providing an electrode;
(b) attaching to said electrode a first oligonucleotide probe, at least a portion of which is complementary to a first end sequence of a nucleic acid;
(c) contacting the electrode of step (b) with said nucleic acid, thereby allowing said first probe to bind said nucleic acid;
(d) providing a second semiconductor nanoparticle to which has been attached a second oligonucleotide probe, at least a portion of which is complementary to a second end sequence of said nucleic acid,
(e) contacting the electrode of step (c) with said second nanoparticle, thereby allowing said second probe to bind said bound nucleic acid;
(f) providing a first semiconductor nanoparticle to which has been attached said first oligonucleotide probe and pre-incubating said first nanoparticle with said nucleic acid, thereby allowing said first probe to bind said nucleic acid;
(g) contacting the electrode of step (e) with said pre-incubated first nanoparticle, thereby allowing said nucleic acid bound to said first probe to bind said second probe on said second nanoparticle; and
(h) optionally alternately repeating steps (e) and (g) one or more times.

19. A method for fabricating a semiconductor nanoparticle electronic circuit comprising electron mediator functionalized nucleic acid comprising:
(a) providing an electrode;
(b) attaching to said electrode a first oligonucleotide probe, at least a portion of which is complementary to a first end sequence of a nucleic acid;
(c) contacting the electrode of step (b) with said nucleic acid, thereby allowing said first probe to bind said nucleic acid;
(d) providing a second semiconductor nanoparticle to which has been attached a second oligonucleotide probe, at least a portion of which is complementary to a second end sequence of said nucleic acid;
(e) contacting the electrode of step (c) with said second nanoparticle, thereby allowing said second probe to bind said bound nucleic acid;
(f) providing a first semiconductor nanoparticle to which has been attached said first oligonucleotide probe and pre-incubating said first nanoparticle with said nucleic acid, thereby allowing said first probe to bind said nucleic acid;
(g) contacting the electrode of step (e) with said pre-incubated first nanoparticle, thereby allowing said nucleic acid bound to said first probe to bind said second probe on said second nanoparticle;
(h) optionally alternately repeating steps (e) and (g) one or more times; and
(i) incubating said electrode with an electron mediator capable of binding nucleic acids.

20. A method for fabricating a semiconductor nanoparticle electronic circuit comprising semiconductor arrays crosslinked by nano metallic rods comprising:
(a) providing an electrode;
(b) attaching to said electrode a first oligonucleotide probe, at least a portion of which is complementary to a first end sequence of a nucleic acid;
(c) contacting the electrode of step (b) with said nucleic acid, thereby allowing said first probe to bind said nucleic acid;
(d) providing a second semiconductor nanoparticle to which has been attached a second oligonucleotide probe, at least a portion of which is complementary to a second end sequence of said nucleic acid;
(e) contacting the electrode of step (c) with said second nanoparticle, thereby allowing said second probe to bind said bound nucleic acid;
(f) providing a first semiconductor nanoparticle to which has been attached said first oligonucleotide probe and pre-incubating said first nanoparticle with said nucleic acid, thereby allowing said first probe to bind said nucleic acid;
(g) contacting the electrode of step (e) with said pre-incubated first nanoparticle, thereby allowing said nucleic acid bound to said first probe to bind said second probe on said second nanoparticle;
(h) optionally alternately repeating steps (e) and (g) one or more times; and
(i) incubating said electrode with a metal capable of binding nucleic acids.

21. A method according to claim 1 wherein said semiconductor nanoparticle comprises CdS and said nucleic acid is DNA.

22. A semiconductor device comprising a dendritic nanoparticle array comprising semiconductor nanoparticles cross-linked by nucleic acid chains.

23. A system for identifying a target nucleic acid sequence in a sample comprising:
(a) a biochip comprising a plurality of arrays of functionalized solid surfaces each of which may act as a transducer, each of the surfaces having bound thereto an oligonucleotide probe, at least a portion of which is complementary to a different segment of a target nucleic acid sequence, each of the arrays being specific for a different target nucleic acid sequence; and
(b) semiconductor nanoparticles functionalized with oligonucleotide probes, at least a portion of which is complementary to one end sequence or the other end sequence of one of the target nucleic acid sequences.

24. A system according to claim 23 wherein said different target nucleic acid sequences are nucleic acid sequences of different pathogenic microorganisms.

25. A system according to claim 23 wherein said different target nucleic acid sequences are nucleic acid sequences related to different genetic diseases.

26. A system according to claim 23 wherein said different target nucleic acid sequences are nucleic acid sequences of different tissues.

27. A system according to claim 23 wherein said different target nucleic acid sequences are nucleic acid sequences of different individuals.

28. A kit for the detection of a target nucleic acid sequence in a sample containing a mixture of nucleic acids comprising:
(a) a functionalized solid surface which acts as a transducer and having a probe attached thereto; and
(b) semiconductor nanoparticles functionalized with oligonucleotide probes, at least a portion of which is complementary to one end sequence or the other end sequence of the target nucleic acid sequence.
